(19) United States
(12) Patent Application Publication
(10) Pub. No.: US 2015/0305810 A1
(43) Pub. Date: Oct. 29, 2015

(54) MICROWAVE BASED ELECTROSURGICAL COAGULATING KNIFE

(71) Applicant: AngioDynamics, Inc., Latham, NY (US)

(72) Inventors: Kevin McElwee, Acton, MA (US); Jeffrey Model, Cambridge, MA (US)

(21) Appl. No.: 14/697,186

(22) Filed: Apr. 27, 2015

Related U.S. Application Data
(60) Provisional application No. 61/984,131, filed on Apr. 25, 2014.

(51) Int. Cl. A61B 18/18; A61B 18/14

(52) U.S. Cl. CPC A61B 18/1815; A61B 18/1402; A61B 2018/00083

(57) ABSTRACT

A microwave based electrosurgical coagulating knife capable of simultaneous or near simultaneous cauterization and dissection of tissue to prevent unwanted or dangerous blood loss during tissue removal.

### CROSS-REFERENCE TO RELATED APPLICATIONS

The present application claims priority to U.S. provisional patent application No. 61/984,131, filed Apr. 25, 2014, which is incorporated herein in its entirety.

### FIELD OF INVENTION

The present invention relates to a medical device and method for treating tissue, and more particularly to removal of tissue via planar coagulation and resection.

### BACKGROUND OF INVENTION

When tissue is to be removed from a human body it is important to reduce the amount of blood loss. In highly vascularized areas of the human body, such as the liver, it is common to first coagulate or segmentally ablate along the incision prior to cutting or removing the tissue.

During tissue resection cases, as shown in FIGS. 1 and 2, sections of tissue 2 are removed via planar coagulation and finally resected so as to minimize bleeding. During planar coagulation, a plane of tissue 6 is coagulated to separate healthy tissue 4 from tissue 8 that's desired to be removed. Once the plane of coagulated tissue 6 has been established by a coagulation device, the physician uses some form of a scalpel to cut along the coagulated plane in order to remove the target tissue 8 from the remaining parenchyma. The coagulated plane of tissue 6 should be within a desired width in order to avoid blood loss from surrounding healthy tissue 4. The depth and length of the coagulated plane is often dependent upon the coagulation device's geometry and the individual patient. If it is desired to resect tissues that have a depth greater than which a single pass of the coagulation device can coagulate, the physician will typically coagulate a portion of the desired plane with the coagulation device, resect the coagulated region of desired plane by a resecting device until uncoagulated tissue area is reached, and repeat the coagulation and resection by switching between the coagulation device and resecting device. This procedure would be repeated until the entire depth of the desired coagulation is complete.

As can be seen above, having two separate devices for coagulation and resection results in a long procedure time, especially when the devices have to be switched back and forth several times. If the physician attempts to shorten the procedure time, incomplete coagulation may occur which results in bleeding post-resection. As shown in FIG. 3, the tissue still has vasculature with blood escaping along the dissection line.

Therefore, it would be desirable to provide a device and method for a single device to coagulate and resect the tissue.

### SUMMARY OF THE DISCLOSURE

According to one aspect of the present invention, an electrosurgical coagulating knife that allows simultaneous or near simultaneous coagulation and resection is provided. The knife includes a blade for cutting tissue and a microwave antenna positioned near the blade and adapted to transmit microwave signal to coagulate the tissue.

According to another aspect of the present invention, a method for performing resection of tissue is provided. A combination electrosurgical coagulating and resecting knife is placed near the tissue to be cut. The knife has a blade for cutting the tissue and a microwave antenna adapted to transmit a microwave signal. After the knife has been placed near the tissue, a microwave signal from the microwave generator is transmitted to the microwave antenna to coagulate the tissue to be cut. When the tissue has been coagulated, the tissue is cut by the blade.

Advantageously, the present invention allows a physician to both coagulate and resect the tissue with a single device without switching between multiple devices, thereby saving procedure time and improving patient safety.

### BRIEF DESCRIPTION OF THE DRAWINGS

FIG. 1 depicts a partial cross-section side view of the ablation zone, specifically the horizontal and vertical planes of the coagulated tissue inside of the non-target tissue.

FIG. 2 depicts a top view of the coagulated plane inside of the non-target tissue.

FIG. 3 depicts a picture of dissected tissue that was not completely coagulated post resection.

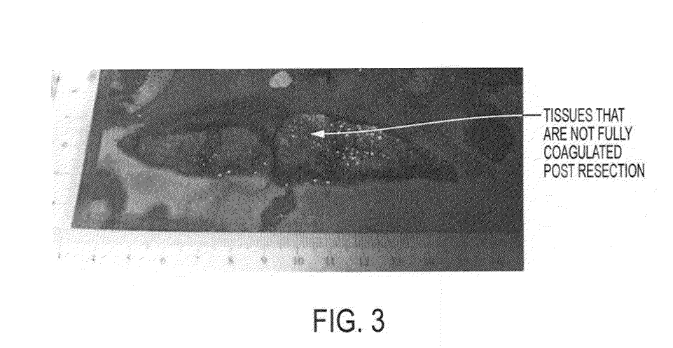

FIG. 4 depicts a microwave based coagulating knife system according one aspect of the present invention. This figure depicts a microwave antenna inside the blade or scalpel connected to a microwave energy cable or wire. The blade or scalpel also comprises a sensor or resistor to measure impedance. The device may also include a first and second notification elements, depicted here as green or red LEDs.

FIG. 5 depicts a first and second notification elements, depicted here as green or red LEDs, and intended to help guide user with the speed or rate of dissection to ensure the dissected tissue is properly coagulated prior to dissection.

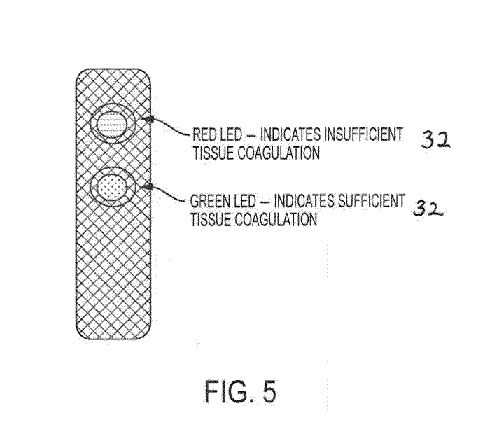

### DETAILED DESCRIPTION OF THE INVENTION

The following descriptions and the associated drawings describe exemplary embodiments in the context of certain exemplary combinations of elements and/or functions, it should be appreciated that different combinations of elements and/or functions can be provided by alternative embodiments without departing from the scope of the appended claims. In this regard, for example, different combinations of elements and/or functions than those explicitly described above are also contemplated.

The first embodiment of this device may have wires or other energy transducers attached to the scalpel or blade near the distal end of the device. This scalpel or blade may act as an electrode. For example, if microwave energy is being used the scalpel or blade may act as a microwave antenna capable of transmitting microwave energy required to coagulate or segmentally ablate along the incision line. The focus or radiating portion of the antenna can be altered and controlled so microwave energy is delivered prior to the blade contacting the tissue. Alternatively, the scalpel/blade with low thermal conductivity can absorb the microwave energy to convert the energy into heat. In that case, the heated blade assists in thermally coagulating tissue in combination with the transmitted microwave energy being absorbed by the tissue. In a particular embodiment, the heated blade by itself may be sufficient to coagulate the tissue when the blade comes in contact with the tissue.

Currently in the art, doctors first cauterize along the incision line and then follow this procedure with the actual incision or dissection of tissue—leading to a time consuming process requiring the patient to be sedated for a longer period of time. By incorporating a microwave antenna near the sharp tip/edge of the blade or scalpel, it increases the efficiency of the current technology found in the art by partially ablating, closing or cauterizing blood vessels in the path of the intended dissection line, thereby preventing unwanted or dangerous blood loss during the actual dissection.

Alternative embodiments may include a sensor on the device capable of detecting when the microwave energy has completed the cauterization. The device may also include a notification element notifying the user that it is safe to dissect or make the intended incision. Also, it is conceived that the device may comprise another sensor capable of detecting the difference between target tissue (such as cancerous tissue) and health tissue. This sensor may be connected to a notification element (such as LED's or noise generator) to notify the user that the dissection line is free from target cells, thereby confirming that the tissue being removed contains all of the intended target tissue that needs to be removed. The advantage to such an embodiment is that the user will have confirmation that no target tissue remains in the patient. This is beneficial because it obviates the need for the patient to endure another procedure if the target tissue containing cancer cells is not completely removed and the cancer cells spread to the healthy tissue. As can be appreciated, using such a device is beneficial for patients because often they may not be able to tolerate any more surgical procedures.

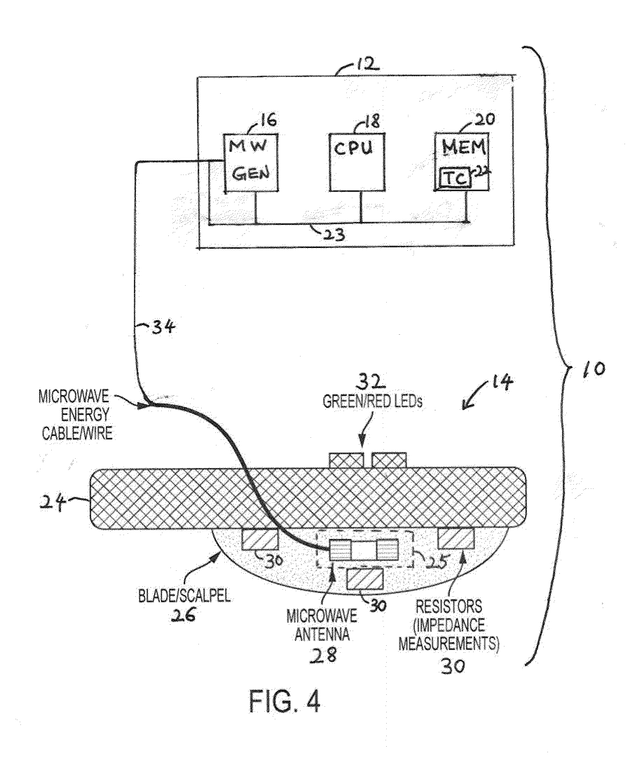

According to one aspect of the present invention as shown in FIG. 4, an electrosurgical coagulating knife system 10 may perform the acts of coagulation and resection simultaneously or near simultaneously, thereby substantially reducing the time of the procedure.

The system 10 includes a treatment control computer 12 and a coagulating knife 14. The treatment control computer 12 has a microwave generator 16, a processor (CPU) 18 and memory 20. The memory 20 stores a treatment control program/module 22 that controls the microwave generator 16 and the processor 18 to regulate the coagulation and resection of tissue. The memory 20, microwave generator 16 and the processor 18 are all coupled to each other through a common bus 23.

The coagulating knife 14 has a handle 24, a cutting blade 26 attached to the underside of the handle, a microwave antenna 28 and coagulation sensors 30 attached to the blade and visual indicators 32 positioned on the handle. The microwave antenna 28, coagulation sensors 30 and visual indicators 32 are connected to the treatment control computer 12 by wires in an electrical cable 34. The wires from the microwave generator 16 is connected to the microwave antenna 28 while the wires for the coagulation sensors 30 and visual indicators 32 are connected to the bus 23.

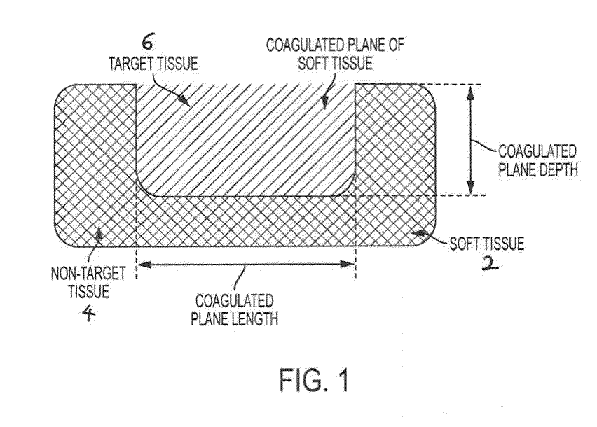

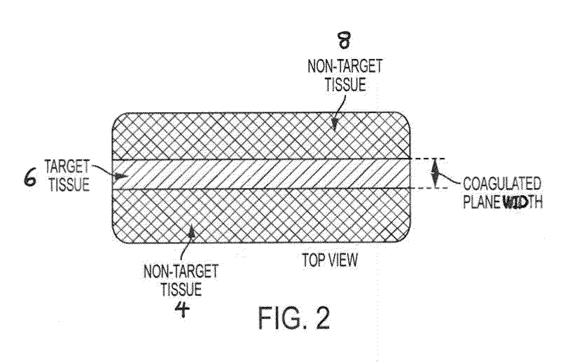

Under the control of the processor 18 and treatment control program 22, the microwave generator 16 generates microwave signal and transmits it through the electrical cable 34. The microwave antenna 28 receives the microwave signal through the cable 34 and emits/radiates it towards the target tissue in a relatively narrow plane as illustrated in FIGS. 1 and 2. The emitted microwave signal generates heat in a known manner and thermally coagulates the tissue in a very short time.

A treatment control module 22 stored in the memory 20 works in conjunction with the processor 18 to control the microwave generator 16 and the visual indicators 32. The processor 18, under the control of the treatment control module 22, receives signals from the coagulation sensors 30 and determines whether sufficient coagulation has taken place based on the received signals. The coagulation sensor can be an impedance sensor such as high precision resistors that can detect the resistance of the tissue. As the tissue coagulates and dries, the resistance of the tissue increases. When the resistance level reaches or rises past a predetermined threshold resistance level (or decreases below a predetermined threshold conductivity level), the processor 18 determines that sufficient coagulation has taken place.

When tissue coagulates, its resistance increases. Blood is conductive and thus has lower electrical resistance/impedance. Therefore, using an impedance measurement feedback can assist in determining whether there is sufficient coagulation and/or minimal blood loss during the resection process. A specific resistance measurement level would be used to continuously determine whether or not sufficient coagulation has occurred (Rcoag). If the measured resistance is less than Rcoag, then the first notification element, such as a red LED 32, is activated. This would visually indicate that the physician is either (a) moving the knife 14 too fast and would thus need to slow down the pull back of the blade over the tissue or (b) the blade 26 of the knife 14 is too deep within the tissue and the emitted microwave energy cannot sufficiently ablate the adjacent tissues.

If the measured resistance is greater than or equal to Rcoag, then the second notification element, such as a green LED 32, would activate, visually indicating that the pullback speed of the blade 26 and depth of the blade relative to the tissue are sufficient for soft tissue coagulation with minimal blood loss. If the width of the coagulated plane is too small, excess blood loss can occur during resection. For these reasons, a method of measuring the magnitude of coagulated tissues adjacent to the blade is helpful.

The visual indicators 32 can be used to visually indicate the coagulation status to the physician. For example, the indicators 32 can consist of a green LED and a red LED. The processor 18 can light up the red LED 32 to indicate that that coagulation has not completed. When sufficient coagulation of the tissue has been determined by the processor 18, it can send a signal to light the green LED 32.

In addition to visual indicators 32 the device may alternatively include audio indicators (not shown). The audio indicators may provide various pitches or tones to indicate to the user if proper coagulation has occurred. For example, if the measured resistance is less than Rcoag, the audio indicators may produce a low pitch audio sound, or a beeping audio sound. This may indicate to the user that the coagulation is not sufficient. If the measured resistance is greater than or equal to Rcoag, the audio indicators may produce a high pitched audio sound, or a constant non-interrupted audio sound, indicating that sufficient coagulation has occurred.

When the processor 18 determines that sufficient coagulation has taken place, it can also send a signal to the microwave generator 16 to either turn off the microwave signal or reduce its power. The processor 18 continues to monitor the coagulation status through the coagulation sensor 30. If the resistance starts to decrease below a predetermined resistance level (above a predetermined conductivity level), it indicates that the blade has moved to a different position within the target tissue. The processor 18 can then switch off the green LED 32 and turn on the red LED 32 while at the same time send a signal to the microwave generator 16 to turn on the microwave signal or increase its signal.

Microwave energy reflects off metals. If the microwave antenna 28 were orientated such that the energy is emitted away from the blade 26, a metal blade could be sufficient. The blade 26 could also be made of a material that allows microwave energy to pass through it very well in order to reduce the amount of reflected energy (e.g., ceramic). Reducing the amount of reflected energy would increase the amount of energy delivered to the tissue. The electric field lines produced by the emitting antennae would appear as so while the device is turned ‘ON’.

The blade 26 can be made of either non-conductive material such as ceramic or conductive material such as aluminum or steel.

If the blade 26 is conductive and has a relatively high thermal conductivity (e.g., thermal conductivity W/(m K) at 25 degree Celsius of 75 or more, preferably 100 or more), the blade will reflect the microwave energy without absorbing it. This way, the microwave antenna 28 can be designed (with optional reflectors 25) to direct the microwave signal laterally away from the blade 26 to create a thermal zone which is long (along the length of the blade) but with a narrow width (lateral to the blade) which closely matches the coagulated plane 6 depth and width as shown in FIGS. 1 and 2.

The microwave antenna 28 can also be orientated in such a way as to emit microwave energy from a plane (i.e., micro strip antenna) rather than a single point (e.g., wire antenna). This plane of microwave emission can also better assist the physician in sealing any bleeding vessels once the desired tissue has been resected.

Alternatively, the blade 26 can be designed with material with a relatively low thermal conductivity (e.g., thermal conductivity W/(m K) at 25 degree Celsius of less than 75, preferably 50 or less) and generally non-conductive material such as ceramic material. Such material will absorb the microwave energy emitted from the microwave antenna 28. In that case, the blade 26 can be used as part of the heat source to thermally coagulate the tissue or can even be sufficiently hot to coagulate the tissue by itself. For enhanced heating, metal powders of less than 200 microns (preferably 100 microns or less) in size can be uniformly added to the blade 26.

If the tissue is resected and has not fully or completely coagulated, it can emit fluids such as blood. The microwave antenna 28 of the above embodiments is intended to completely seal the tissue plane along the resection line to prevent any fluid post coagulation. In the embodiments in which the device 10 has the capability of emitting microwave energy from a plane (e.g., the surface area of a blade), it may better assist the user in completely coagulating these tissue that were not yet fully coagulated even after resection.

The knife 14 can act as a planar coagulation tool and scalpel—it coagulates a plane of tissue while cutting the coagulated tissues. Microwave energy is emitted from the microwave antenna 28, which are located on the surface of the scalpel/blade 26. If the rate of coagulation is too fast, excess blood loss may occur. For that reason, the physician should know at what rate to drag the scalpel/blade 26 across the tissues. Conversely, if the rate of coagulation is initially too slow, charring can occur at the blade site, resulting in a reduction of microwave energy being emitted and could prolong the time required to coagulate tissues that are located radially outwards from the blade 26. The red and green LEDs' 32 as well as the microwave signal level controlled by the processor 18 could assist the physician with the appropriate rate of blade 26 movement for a simultaneous or near simultaneous coagulation/resection of tissue.

For example, the red LED 32 may indicate that the user is cutting the target tissue with the knife 14 too slowly and is in danger of charring building up on the knife 14. The green LED 32 may indicate to the user that the knife 14 is moving at the correct speed to produce proper coagulation and no charring.

A method for performing resection of tissue will now be described. A physician places a combination electrosurgical coagulating and resecting knife 14 near the tissue to be cut. After the knife 14 has been placed near the tissue, the microwave generator 16 transmits a microwave signal to the microwave antenna 28 to coagulate the tissue to be cut. The microwave signal generation can be done manually by the physician or automatically by the processor 18 under the control of the treatment control module 22 based on the signal from the coagulation sensors 30. The user may use a foot pedal (not shown) to activate the microwave generator 16 through the treatment control module 22.

When the tissue has been determined to be sufficiently coagulated by the processor 18, it turns off the red LED 32 and turns on the green LED 32 to visually indicate to the physician that the tissue has been coagulated. The physician then moves the blade 26 to cut the tissue.

The processor 18 continually monitors the coagulation status of tissue through the coagulation sensors 30. When the blade 26 moves to a different location of tissue, the processor 18 senses the reduced resistance from the coagulation sensors 30 and turns off the green LED 32 and turns on the red LED 32 visually advising the physician to wait until the coagulation of tissue has taken place. The processor 18 can also turn on or increase the microwave signal automatically.

Once the green LED 32 turns on, the physician can continue to cut the tissue with the blade 26.

The processor 18 can control the transmission of the microwave signal to the microwave antenna 28 from the microwave generator 16 based on sensor signals from the coagulation sensors 30. For example, the processor 18 can turn off or reduce the microwave signal to the microwave antenna 28 when it determines that the sufficient coagulation has taken place.

Depending on the type of blade material used, the method can also sufficiently heat the blade 26 so that it functions as both a cutting blade and a coagulating device.

This completes the description of the selected embodiments of the invention. Those skilled in the art may recognize other equivalents to the specific embodiments described herein which equivalents are intended to be encompassed by the claims attached hereto.

What is claimed is:

1. An electrosurgical coagulating knife comprising:
a blade for cutting tissue;
a microwave antenna positioned near the blade and adapted to transmit microwave signal to coagulate the tissue.

2. The electrosurgical coagulating knife of claim 1, further comprising a coagulation sensor positioned near the blade for sensing the coagulation of tissue by the microwave antenna.

3. The electrosurgical coagulating knife of claim 2, wherein the coagulation sensor includes an impedance sensor for sensing the impedance of the tissue.

4. The electrosurgical coagulating knife of claim 2, further comprising a processor adapted to control the transmission of a microwave signal to the microwave antenna based on sensor signals from the coagulation sensor.

5. The electrosurgical coagulating knife of claim 2, further comprising a processor adapted to receive signals from the coagulation sensor and determine whether sufficient coagulation has taken place.

6. The electrosurgical coagulating knife of claim 4, wherein the processor is programmed to turn off or reduce the microwave signal to the microwave antenna when the processor has determined that sufficient coagulation has taken place.

7. The electrosurgical coagulating knife of claim 1, further comprising a coagulation indicator positioned near the blade and operable to visually indicate that sufficient coagulation has taken place.

8. The electrosurgical coagulating knife of claim 1, wherein the blade is non-conductive and is adapted to convert the microwave signal from the microwave antenna into heat sufficient to coagulate the tissue.

9. The electrosurgical coagulating knife of claim 1, wherein:
the blade is non-conductive; and
the microwave antenna is adapted to transmit the microwave signal to the tissue for heating the tissue and to the blade for heating the blade, wherein the combination of the heated blade and heated tissue coagulate the tissue.

10. The electrosurgical coagulating knife of claim 1, wherein the blade is sufficiently conductive to reflect the transmitted microwave signal to the tissue for coagulation.

11. The electrosurgical coagulating knife of claim 10, wherein the blade includes metal.

12. A combination electrosurgical coagulating and resecting knife comprising:
a blade for cutting tissue;
a coagulation sensor positioned near the blade for sensing the impedance of the tissue;
a microwave antenna positioned near the blade and adapted to transmit a microwave signal to coagulate the tissue so as to allow the blade to cut the tissue without bleeding; and
a processor adapted to receive signals from the coagulation sensor and determine whether sufficient coagulation has taken place based on the received signals.

13. A method for performing resection of tissue comprising:
placing a combination electrosurgical coagulating and resecting knife near a tissue to be cut, the knife having a blade for cutting the tissue, and a microwave antenna positioned near the blade and adapted to transmit a microwave signal;
after the knife has been placed near the tissue, transmitting a microwave signal to the microwave antenna to coagulate the tissue to be cut;
cutting the tissue by the blade when the tissue has been coagulated.

14. The method of claim 13, further comprising sensing, using a coagulation sensor positioned near the blade, the coagulation of tissue by the microwave antenna.

15. The method of claim 14, wherein the step of sensing includes using an impedance sensor for sensing the impedance of the tissue.

16. The method of claim 14, further comprising controlling the transmission of a microwave signal to the microwave antenna based on sensor signals from the coagulation sensor.

17. The method of claim 14, further comprising:
receiving sensor signals from the coagulation sensor; and
determining whether sufficient coagulation has taken place based on the received sensor signals.

18. The method of claim 16, wherein the step of controlling includes turning off or reducing the microwave signal to the microwave antenna when it has been determined that the sufficient coagulation has taken place.

19. The method of claim 13, further comprising observing a coagulation indicator positioned near the blade and visually indicating that sufficient coagulation has taken place.

20. The method of claim 19, further comprising cutting the tissue with the blade when the coagulation indicator indicates that sufficient coagulation has taken place.

21. The method of claim 13, wherein the blade is non-conductive and the step of transmitting includes transmitting the microwave signal toward the blade to sufficiently heat the blade for coagulating the tissue.

22. The method of claim 13, wherein the blade is non-conductive and the step of transmitting includes transmitting the microwave signal toward the tissue for heating the tissue and toward the blade for heating the blade, wherein the combination of the heated blade and heated tissue coagulate the tissue.

23. The method of claim 13, wherein the blade is conductive and reflects the transmitted microwave signal toward the tissue for coagulation.
